(19) United States
(12) Patent Application Publication
(10) Pub. No.: US 2008/0275533 A1
(43) Pub. Date: Nov. 6, 2008

(54) DISPLAY APPARATUS FOR PROVIDING INFORMATION AND THERAPEUTIC LIGHT

(76) Inventor: Steven D. Powell, Orem, UT (US)

Correspondence Address: STOEL RIVES LLP - SLC, 201 SOUTH MAIN STREET, ONE UTAH CENTER, SALT LAKE CITY, UT, 84111, US

(21) Appl. No.: 11/744,734

(22) Filed: May 4, 2007

(51) Int. Cl. A61N 5/06

(52) U.S. Cl. 607/88

(57) ABSTRACT

The invention relates to a light therapy device that may be used in the treatment of various conditions. The light therapy device may include a display screen communicating one or more operating parameters to the user. Furthermore, the light therapy device may include a control system that receives, from a user, an indication of the ailment or condition to be treated. The user input can then be used to determine how the condition may be treated. Therapeutic light from a light source, such as an array of LEDs, may be directed through the display screen to a person receiving the treatment.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a top view of an embodiment of an LED array for use in connection with a display screen of a light therapy device;

FIG. 2 is a cross-sectional view of the light therapy device display screen shown in FIG. 1;

FIG. 3 is a block diagram of a system for treating various conditions with light;

FIG. 4 is a perspective view of an embodiment of a light therapy device including a touch-screen control; and

FIG. 5 is a perspective view of another embodiment of a light therapy device including a push-button control.

### DETAILED DESCRIPTION

Reference is now made to the figures in which like reference numerals refer to like elements. While the various aspects of the embodiments disclosed are presented in drawings, the drawings are not necessarily drawn to scale.

Those skilled in the art will recognize that the systems and methods disclosed can be practiced without one or more of the specific details, or with other methods, components, materials, etc. In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the figures herein could be arranged and designed in a wide variety of different configurations.

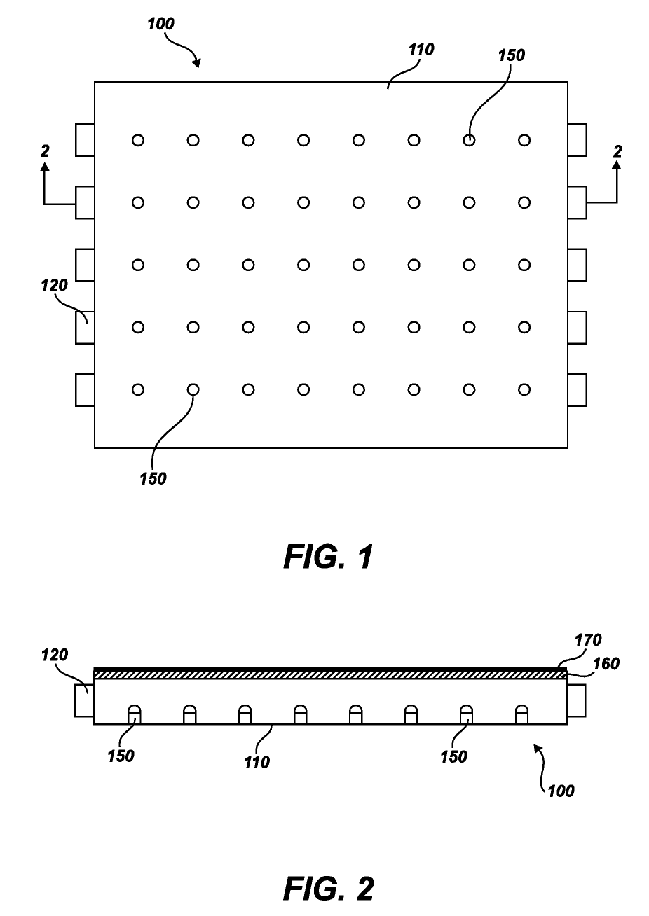

FIG. 1 represents one embodiment of a light array 100 that may be used in the treatment of various diseases or conditions, as described more fully below. FIG. 2 shows the light array 100 as a cross-section at the location indicated in FIG. 1. More particularly, FIG. 1 and FIG. 2 show a light array 100 with at least one light source, such as an array of light emitting diodes (LEDs) 150 configured to project light through a display screen 170.

The display screen 170 may be disposed on a housing 110 that may be shaped as desired, such as in a rectangular, circular, spherical, or other desired shape sufficient to house the light array 100. As described in greater detail below, the display screen 170 may be used to input parameters for controlling the light array 100, e.g., wavelength, duration, condition to be treated, etc, as well as to display the parameters or other information when the light array 100 is not in use.

In one embodiment, the light array 100 may also include one or more power modules 120 configured to direct and control an electric current to the light array 100. The light array 100 may be powered by an internal or external portable power source, such as a battery. The battery power source may provide the LEDs 150 with power such that AC power is not required. Alternatively, an AC adapter or direct AC connection may be used in other embodiments. The batteries may be stored within or proximate to the light array 100 and electrically connected to one or more of the power modules 120 to provide energy for the light array 100.

The array of LEDs 150 is located such that the light emitted from the each of the LEDs 150 may be directed to pass through the surface of the display screen 170 and towards a user located in front of the display screen 170. The light therapy device 100 may also include a light diffusing screen 160 disposed with the display screen 170 that may be configured to diffuse the light from the light array 100 as it passes through the display screen 170. The light array 100 may include one or more light sources of white light or other wavelengths of light such as colored light emitted from an array of colored LEDs.

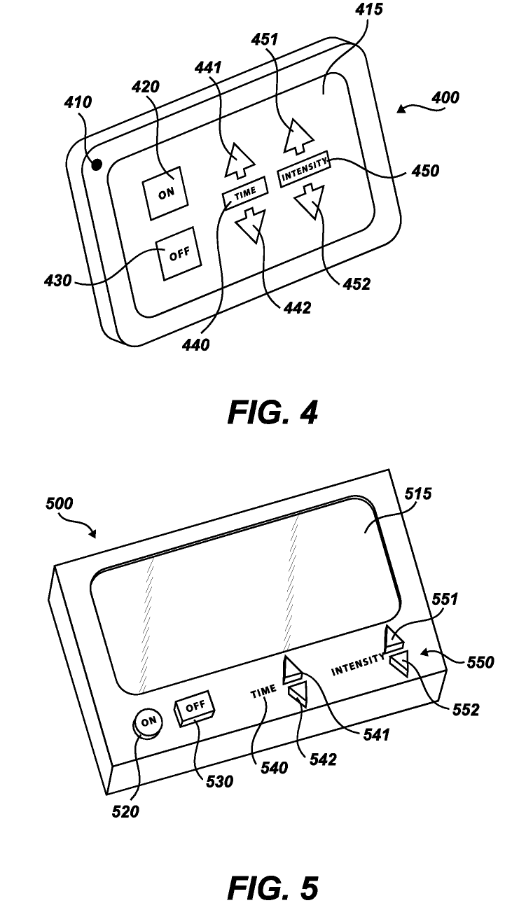

The light array 100, including the array of LEDs 150, may be activated when a user depresses a control area on the display screen 170, which may be configured as touch screen. For example, FIG. 4 shows a light therapy device 400 which may have a touch screen 415 with touch-sensitive controls. The touch screen 415 may be a liquid crystal display (LCD) screen of the type used for touch-sensitive controls as known in the art. The touch-sensitive controls may be fixed or movable (“soft”). For instance, the touch-sensitive controls may be permanently marked on the surface of the display screen 170, such that activation of a particular control will always be registered in response to the user touching a corresponding location of the display screen 170. In an alternative embodiment, the touch-sensitive controls may be graphical images programmed to be displayed in different areas of the display screen 170 as desired. Alternatively, switches, membrane buttons, voice activation and recognition, or the like, may be used to receive user input as would be apparent to those having skill in the art.

The light array 100 may be positioned behind the touch screen 415 in order to transmit therapeutic light through the touch screen 415. When the user chooses to activate the light therapy device 400, the display screen 415 may be started by activating a control button, such as control button 410 disposed on the light therapy device 400. When the display screen 415 is active, a user may operate the light therapy device 400 by touching the control surfaces on the display screen 415. For example, the control surfaces may be operated with the user's finger or with a stylus. More particularly, a user may touch the ON control 420 to turn on a light source, like the light array 100 shown by FIGS. 1 and 2, disposed behind the touch screen 415, thereby directing a therapeutic light through the touch screen 415 and towards the user. The OFF control 430 may be used to terminate the light therapy and TIME controls 441 and 442 may be used to increase or decrease the duration of the treatment. The display screen 415 may include other control parameters, such as INTENSITY controls 451 and 452, which may be used to control the intensity or wavelengths of the light source shining through the display screen 415. Alternatively, a separate WAVELENGTH control (not shown) may be provided. In still another embodiment, a CONDITION control (not shown) may be provided, which allows a user to select from a variety of conditions to treat, in which case the device 400 will automatically select an optimal wavelength and/or intensity from an internally stored table or the like. The display screen 415 may also display other information and data such as the date and time, the currently selected control parameters, etc.

The light therapy device 400 may be included as a part of many common consumer devices such as mobile cellular phones, personal digital assistants, portable video game consoles, portable music devise, and other portable devices with a display screen 415 through which a therapeutic light may be directed towards a user.

The light source may also be activated when a user pushes a button or switch. For example, FIG. 5 shows a light therapy device 500 which may include a housing 550 and control buttons for allowing a user to operate the device. The housing 550 may be configured to house a display screen 515 disposed and a light source, the display screen disposed over the light source. The display screen 515 may be a flat panel of glass or plastic that can be transparent or at least partially opaque. The display screen 515 may be configured to allow the light from the light source, such as an array of LEDs as shown in FIGS. 1 and 2, to pass through and be directed towards a user. The control buttons may be labeled by marking the housing, such as the TIME label 540 next to the time control buttons 541 and 542. The control buttons may also be directly labeled such as show by the ON button 520 and the OFF button 530.

When the user chooses to activate the light therapy device 500, it may be started by activating a control button, such as the ON button 520 which can be in electric communication with a control element of the light source housed behind the display screen 515. More particularly, a user may push the ON button 520 to turn on the light source disposed behind the display screen 515, thereby directing a therapeutic light through the display screen 515 and towards the user. The OFF button 530 may be pressed to turn the light source off and the TIME buttons 541 and 542 may be used to increase or decrease the duration of the light therapy treatment. The light therapy device 500 may include other control buttons, such as INTENSITY buttons 551 and 552, which may be used to control the intensity or wavelengths of the light source shining through the display screen 515. The display screen 515 may display the control information and data such as the time value and the intensity value, as well as the date and time, etc.

With reference to FIGS. 1 and 2, the light source may emit a broad or narrow spectrum of light. For purposed of example only, and not meant as a limitation, the light source may include the array of LEDs 150 that emit light in a narrow range of wavelengths. Since the LEDs 150 emits a narrow range of wavelengths, often the light emitted is considered monochromatic. LEDs 150 typically use less power, produce less heat, and have a longer life span than most incandescent lamps. Furthermore, LEDs 150 are often an inexpensive alternative to wavelength selection compared to lamp and filter systems. Furthermore, the compactness and portability of an LED light therapy device, such as light therapy devices 400 and 500, are typically superior to alternative lamp and filter designs.

According to one embodiment, LEDs 150 include one or more multi-color LEDs in a single LED package, which is capable of emitting more than one discrete range of wavelengths. For example, in one embodiment the multi-color LEDs 150 can include a bi-color, or bi-polar LED producing two discrete ranges of wavelengths. As such, individual LEDs may produce a narrow band of wavelengths in the red portion of the visible electromagnetic spectrum as well as a narrow band of wavelengths in the blue portion of the visible electromagnetic spectrum. The red wavelengths may range between approximately 630 nanometers and 680 nanometers, while the blue wavelengths may range between approximately 400 nanometers and 470 nanometers. In one embodiment, the red band is between approximately 650 to 670 nanometers and the blue band is between approximately 405 to 420 nanometers.

One or more of the multi-color LEDs 150 may be capable of producing only red wavelengths at one time, or only blue wavelengths, or both red and blue wavelengths simultaneously. In other embodiments, the multi-color LEDs 150 can include at least one tri-color LED producing three discrete ranges of wavelengths. As would be apparent to those having skill in the art, multi-color LEDs 150 may be used which can produce more than three discrete wavelengths as the advancement of technology permits.

The light therapy devices 400 and 500 of FIGS. 4 and 5 may be used to treat a variety of conditions. For example the display screens 415 and 515 may be directed toward or placed on a region of skin having a particular skin condition so that the skin may be treated with LED light therapy. The light therapy devices 400 and 500 may be small and portable so that small focused light may be directed, for example, around the eyes of a user or other small specific areas where conditions may exist that larger light therapy devices may not be able to treat.

The light therapy devices 400 and 500 can produce specific wavelengths that may be therapeutic for a number of ailments or conditions. For example, for the treatment of acne, both blue wavelengths (400 to 470 nanometers) and red wavelengths (630 to 680 nanometers) may be used. Furthermore, for the treatment of acne, the light therapy devices 400 and 500 may provide twice as much exposure to blue wavelengths than to red wavelengths in a single treatment event. Relative exposures of red and blue wavelengths may be determined through a quantifiable value such as light intensity or duration of exposure.

In order to treat wrinkles in the skin, blue, red and yellow wavelength bands may be used. The blue and red wavelength ranges are approximately 400 to 470 nanometers and 630 to 680 nanometers, respectively. The yellow band of wavelengths may be between approximately 530 nanometers and 600 nanometers.

In treating rosacea, a yellow range of wavelengths may be used between approximately 530 and 600 nanometers.

In treating sun spots, a yellow range of wavelengths (530 to 600 nanometers) may be used. For alternative forms of sun damage, a red band (630 to 680 nanometers) may be employed.

Blue light (between 400 and 470 nanometers) may be used to treat and kill bacteria that may cause various forms of skin blemishes, such as acne.

Inflammation may be treated by exposing affected skin to red wavelengths (630 to 680 nanometers) and also to infrared wavelengths, which may range from about 800 nanometers to about 1000 nanometers. As discussed above, the two wavelength ranges may be produced by a single multi-color LED or by two separate LEDs, or an array of LEDs 150 as would be apparent to those having skill in the art.

Lesions in the skin may be treated by illuminating the affected area with red wavelengths (630 to 680 nanometers) and infrared wavelengths (800 to 1000 nanometers).

Canker sores may also be treated by irradiating the sore to red and infrared wavelengths (630 to 680 nanometers and 800 to 1000 nanometers, respectively). A typical one time treatment of canker sores may have a duration of exposure between 5 and 15 minutes, with an intensity of approximately 105 mW/cm². However, multiple applications may be necessary in certain situations.

Skin blemishes may be treated through exposure to red, blue and yellow wavelengths. As discussed above the wavelength ranges may be approximately from 630 to 680 nanometers for red, approximately 400 to 470 nanometers for blue, and approximately 530 to 600 nanometers for yellow.

LEDs 150 that emit a band of wavelengths in the green portion of the visible electromagnetic spectrum may also be used in treating various conditions such as sun spots, rosacea and wrinkles. The wavelength range associated with green light may range between approximately 500 nanometers and 530 nanometers. LED light therapy may also be used in treating dead skin and other skin problems.

In one embodiment, the light therapy devices 400 and 500 of FIGS. 4 and 5 may be used to treat non-skin conditions, such as seasonal affective disorder (SAD), general depression, sleep disorders, shift-work disorders, post- and ante-partum depression, pre-menstrual syndrome, late luteal phase dysphoric disorder (LLPDD), various eating disorders (including bulimia), and chronic fatigue. In such an embodiment, white light may be used and/or blue light having a wavelength range between approximately 440 and 480 nanometers. The foregoing conditions are generally thought to be types of circadian rhythm disorders, the treatment of which involves providing therapeutic wavelengths of light to the patient's eyes. Hence, treatment methods are often referred to as “ocular” light therapy. In one embodiment, the light source may be held close enough to the eyes to achieve effective high-intensity lux output.

The light therapy devices 400 and 500 shown in FIGS. 4 and 5 may also include a lens or diffuser, such as diffuser 160 shown in FIG. 1, to diffuse ultra violet light or other light rays that may be emitted from the light array 100. Furthermore, the LEDs 150 may be removable from the light array 100 and can be replaced with another color LED or another multi-color LED for treatment of a different skin condition.

The light therapy devices 400 and 500 may be configured to be cradled in a recharging base station (not shown). In the cradle position, the base station may have contact points that are in electronic communication with contact points of the light therapy devices 400 and 500. The base station can be connected to an AC power supply through a power cord (not shown). Alternatively, the light therapy devices 400 and 500 may be recharged using an AC adapter.

In one embodiment, the LEDs 150 may include a plurality of red and blue LEDs. In some embodiments, each LED is a single color LED, while in other embodiments, multi-color LEDs may be used. In the single color LED embodiment, the red and blue LEDs may be arranged in a checkerboard configuration, where every other LED emits blue wavelengths while all other adjacent LEDs emit red wavelengths.

Alternatively, other color LEDs may be used, particularly those that are capable of emitting yellow, green and infrared wavelengths. The array of LEDs 150 may also be programmed to emit a combination of wavelengths simultaneously to treat different conditions at the same time. Furthermore, light therapy devices 400 and 500 may also emit different intensities of light. For example, a user may control the intensities of all or some of the LEDs 150 by adjusting a touch-screen display or control buttons. The intensities of each color may also be varied independently.

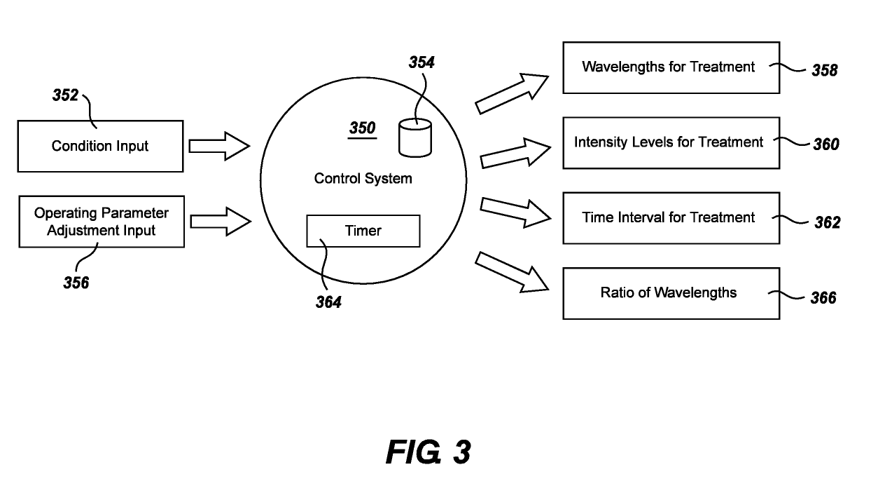

FIG. 3 is a block diagram of a control system 350 for treating various conditions with an LED light therapy device. The control system 350 may be incorporated, in part, into a device controller as, such as a controller for light therapy devices 400 and 500. The control system 350 may receive various forms of user input in order to control various treatment modes of the light therapy device.

For example, a user may provide input 352 indicative of a condition that a user desires to be treated by the LED light therapy device. Examples of various skin condition inputs 352 may include acne, rosacea, wrinkles, inflammation, sun spots or sun damage, bacteria, blemishes, lesions or canker sores. A user may select one or more of a list of conditions to be treated and the control system 350 accesses operating parameters stored on a memory device 354 or database in machine readable form. The operating parameters of the light therapy device that correspond with a particular light therapy treatment may be inputted by a manufacturer or programmer of the device, or alternatively a user may provide adjustment operating parameter input 356 in accordance with a customized treatment program.

The control system 350 accesses the memory device 354 containing multiple operating parameters and selects those corresponding to the condition input 352 received. The light therapy device then runs according to the operating parameters corresponding with the selected condition input 652. One example of an operating parameter output of the control system 350 is a control signal corresponding to the specific wavelengths for treatment 358 of the condition selected. Accordingly, if acne is selected by the user, the control system 350 accesses the corresponding operating parameter that indicates both red and blue wavelengths are to be used for treatment. However, if the user selected rosacea as the skin condition to be treated, the wavelengths for treatment 358 may be in the yellow band (530 to 600 nanometers).

Another form of output of the control system 350 is the operating parameter that indicates the intensity levels 360 for treatment of the condition selected. For example, with the light therapy device disclosed in FIGS. 4 and 5, the intensity levels of a multi-color LED may be 105 mW/cm². Alternately, with the light therapy device disclosed in FIGS. 4 and 5, an intensity level output 360 of 92 mW/cm² may be provided by the control system 350. A user may adjust the intensity level output 360 corresponding to a particular treatment. The user adjusts that particular operating parameter through input 356 indicating an increase or a decrease in intensity to treat more severe or less severe conditions, respectively. Intensity adjustments may be made, for example, in percentage increments such as ±5%, ±10%, ±15%, etc.

Another operating parameter that may be controlled is the time interval for treatment 362. A typical treatment session may last 15 minutes for some conditions. However, treatment for canker sores may be less, such as between 5 and 15 minutes, depending upon the user input. The time interval for treatment 362 may be controlled by a timer 364, which may be embodied, for example, as a Real Time Clock (RTC). Once the condition input 352 is received and the corresponding operating parameters accessed, the indicated time interval 362 is controlled by the timer 364. Once the timer 364 reaches the time interval 362 indicated it automatically shuts off LED emission of the light therapy device.

Additionally, the operating parameters corresponding to a condition input 352 may include wavelength ratio data 366. For example, when acne is selected as the condition to be treated, the operating parameters corresponding with the treatment of acne would indicate that twice as much exposure to blue wavelengths as compared to red wavelengths is desired. Consequently, the wavelength ratio 366 for acne would be 2:1, blue to red. The relative exposures of red and blue wavelengths may be determined through a quantifiable value such as light intensity or duration of exposure. Therefore, blue LED light may be emitted at twice the intensity of red LED light. Alternatively, the exposure time of blue LED light during a particular treatment interval would be twice as long as red LED light. This may be accomplished by pulsating blue LEDs twice as much as red LEDs, or by activating twice as many blue LEDs than red LEDs, or other methods known to those having skill in the art.

Accordingly, a user is able to control the wavelengths emitted, the intensity levels, the time intervals for treatment, and the relative ratio of wavelengths produced by simply selecting a particular condition. By selecting the condition, the control system 350 causes the LED light therapy device, such as light therapy devices 400 and 500, to provide the appropriate colors, intensity, etc., for that condition.

The control system may be in electronic communication with a display, such as display screens 415 and 515 discussed in conjunction with the description of FIGS. 4 and 5. By way of example, the display screens 415 and 515 may show an indication of the condition selected by the user and the associated operating parameters. In some embodiments, the display screens 415 and 515 may show a countdown of time left or time elapsed for the particular light therapy treatment. Furthermore, an audible alert, such as a beep, may let the user know when the treatment event has ended.

While specific embodiments and applications of light therapy devices have been illustrated and described, it is to be understood that the invention claimed hereinafter is not limited to the precise configuration and components disclosed. Various modifications, changes, and variations apparent to those of skill in the art may be made in the arrangement, operation, and details of the devices and systems disclosed.

What is claimed is:

1. A light therapy device, comprising:
at least one display configured to display information to a user of the light therapy device;
at least one light source configured to emit light through the display; and
a housing that contains the light source, such that light emitting from the light source may be directed through the display and towards a user;
wherein the light source is selected to treat a user condition.

2. The light therapy device of claim 1, wherein the light source comprises at least one light emitting diode (LED).

3. The light therapy device of claim 2, wherein the at least one LED is a multi-color LED

4. The light therapy device of claim 3, wherein the at least one multi-color LED is a bi-color LED.

5. The light therapy device of claim 4, wherein the bi-color LED is bi-polar and emits a range of wavelengths in a blue portion of the visible electromagnetic spectrum and a range of wavelengths in a red portion of the visible electromagnetic spectrum.

6. The light therapy device of claim 5, wherein the range of blue wavelengths is between approximately 400 nanometers and 470 nanometers and the range of red wavelengths is between approximately 630 nanometers and 680 nanometers.

7. The light therapy device of claim 3, wherein the multi-color LED is capable of emitting more than two discrete ranges of wavelengths of light.

8. The light therapy device of claim 1, wherein the user condition to be treated comprises a skin condition.

9. The light therapy device of claim 8, wherein the skin condition is selected from at least one of: acne, rosacea, wrinkles, inflammation, sun damage, bacterial infections, blemishes, and lesions.

10. The light therapy device of claim 1, wherein the user condition to be treated comprises a circadian rhythm disorder.

11. The light therapy device of claim 1, wherein the user condition to be treated comprises at least one of a seasonal affective disorder (SAD), a sleep disorder, and depression.

12. The light therapy device of claim 1, wherein the user condition to be treated comprises at least one of a shift-work disorder, post- and ante-partum depression, pre-menstrual syndrome, a late luteal phase dysphoric disorder (LLPDD), an eating disorder, and chronic fatigue.

13. The light therapy device of claim 1, further comprising a control system to control the light source according to operating parameters, the operating parameters including at least one of intensity level of light emission and duration of light emission, such that the user selects the user condition to be treated whereupon the control system controls the light source in accordance with the operating parameters corresponding to treatment of the selected user condition.

14. The light therapy device of claim 13, the operating parameters further comprising wavelength selection.

15. The light therapy device of claim 1, further comprising a plurality of LEDs arranged in an array.

16. The light therapy device of claim 1, wherein the device is a portable device further comprising a rechargeable battery.

17. The light therapy device of claim 1, wherein the display is an LCD screen display.

18. The light therapy device of claim 1, wherein the display is an LCD touch-screen display.

19. The light therapy device of claim 1, further comprising a user control selected from the group consisting of an LCD touch-screen display, at least one control button, and combinations thereof.

20. The light therapy device of claim 1, further comprising a diffuser disposed with the display and configured to diffuse the light from the light source.

21. A light therapy device for the treatment of user conditions, the device comprising:
a display;
a light emitting diode (LED) illumination source capable of producing at least one range of wavelengths of light to be directed toward a user through the display;
a machine readable medium for storing operating parameters of the LED illumination source, the operating parameters corresponding to treatment of user conditions; and
a control system to receive input from the user indicative of a user condition to be treated, such that the control system accesses the operating parameters corresponding to the indicated user condition and the control system controls the LED illumination source in accordance with the corresponding operating parameters;
wherein the operating parameters include at least one range of wavelengths for treatment of each user condition.

22. The light therapy device of claim 21, wherein the display is an LCD screen display configured to display the operating parameters of the LED illumination source.

23. The light therapy device of claim 21, wherein the display is an LCD touch-screen display configured to allow the user to indicate the user condition to be treated by touching the display screen.

24. The light therapy device of claim 21, further comprising a user control in electronic communication with the control system, the user control selected from the group consisting of a touch-screen display, at least one control button, and combinations thereof.

25. The light therapy device of claim 21, wherein the operating parameters further include at least one intensity level of the light produced by the LED illumination source for treatment of the user condition.

26. The light therapy device of claim 21, wherein the operating parameters further include at least one time interval representing a length of time the LED illumination source emits light for treatment of the user condition.

27. The light therapy device of claim 21, wherein the control system comprises a timer which is set according to the at least one time interval of the operating parameters corresponding to the indicated user condition, such that emission of the LED illumination source is automatically discontinued when the at least one time interval has elapsed.

28. The light therapy device of claim 21, wherein the operating parameters further include at least one wavelength range ratio representing how much of a quantifiable value of one range of wavelengths is emitted relative to the quantifiable value of another range of wavelengths.

29. The light therapy device of claim 21, wherein the operating parameters are adjustable by the user.

30. The light therapy device of claim 21, wherein the LED illumination source comprises an array of LEDs.

31. The light therapy device of claim 30, wherein the array of LEDs are arranged in a housing.

32. The light therapy device of claim 21, wherein the LED illumination source comprises at least one multi-color LED.

33. The light therapy device of claim 21, wherein the display is in electronic communication with the control system, the display showing the skin condition inputted by the user.

34. The light therapy device of claim 21, wherein the display is in electronic communication with the control system, the display showing a timer indicating a treatment time.

35. The light therapy device of claim 21, wherein the user condition to be treated comprises a skin condition.

36. The light therapy device of claim 35, wherein the skin condition is selected from at least one of: acne, rosacea, wrinkles, inflammation, sun damage, bacterial infections, blemishes, and lesions.

37. The light therapy device of claim 21, wherein the user condition to be treated comprises a circadian rhythm disorder.

38. The light therapy device of claim 21, wherein the user condition to be treated comprises at least one of a seasonal affective disorder (SAD), a sleep disorder, and depression.

39. The light therapy device of claim 21, wherein the user condition to be treated comprises at least one of a shift-work disorder, post- and ante-partum depression, pre-menstrual syndrome, a late luteal phase dysphoric disorder (LLPDD), an eating disorder, and chronic fatigue.
